(19) United States
(12) Patent Application Publication
(10) Pub. No.: US 2026/0221243 A1
(43) Pub. Date: Jul. 30, 2026

(54) DEVICES, SYSTEMS, AND METHODS FOR ELECTRONIC HEALTH RECORD COLLABORATION

(71) Applicant: ablooMed, LLC, Dekalb, IL (US)

(72) Inventor: Casey Olsen, Dekalb, IL (US)

(73) Assignee: ablooMed, LLC, Dekalb, IL (US)

(21) Appl. No.: 19/396,310

(22) Filed: Nov. 20, 2025

Related U.S. Application Data
(60) Provisional application No. 63/722,687, filed on Nov. 20, 2024.

(51) Int. Cl. G16H 10/60; G16H 80/00

(52) U.S. Cl. CPC G16H 10/60; G16H 80/00

(57) ABSTRACT

A system for electronic health record (EHR) collaboration may include instructions executable by a processing device to: receive EHR data; compare the EHR data to prior version data; update the prior version data to match the EHR data; receive first update data for the EHR data; generate notification data that indicates an update has been proposed, transmit the notification data, receive second update data, modify the prior version data based on the first or second update data, store the modified prior version data or transmit it to an EHR change log database, and transmit the first or second update data to the EHR database or modify the EHR data based on the first or second update data and transmit the EHR data as modified to the EHR database.

### Related Applications

This application claims the benefit of U.S. Provisional Patent App. No. 63/722,687 by Casey Olsen, filed Nov. 20, 2024, and entitled “DEVICES, SYSTEMS, AND METHODS FOR ELECTRONIC HEALTH RECORD COLLABORATION,” and incorporates the entirety of that application herein by reference.

### BACKGROUND

An Electronic Health Record (EHR) includes health records such as a digital version of a patient's paper chart, treatment protocols, and other health-related matters. It is, ideally, a comprehensive, real-time record that makes information securely available to authorized users. A patient EHR may include a patient's medical history, diagnoses, medications, treatment plans, immunization dates, allergies, radiology images, and laboratory test results. An EHR for a medication may include preparation methods, dispensing methods, dosing protocols, storage, etc. In general, EHRs facilitate the sharing of data across different healthcare settings, with the goal of improving the coordination of care among healthcare providers. EHRs also support other care-related activities directly or indirectly through various interfaces, including evidence-based decision support, quality management, and outcomes reporting. By streamlining the EHR build/configuration process, EHRs may enhance the efficiency and accuracy of patient care.

### SUMMARY

In some aspects, a system for electronic health record (EHR) collaboration is described. The system may include a communication device, a memory device coupled to the communication device, and a processing device coupled to the communication device or the memory device. The memory device may store one or more instructions executable by the processing device to: receive EHR data from an EHR database, wherein the EHR data is indicative of an EHR; compare the EHR data to prior version data associated with the EHR data, wherein the prior version data is stored on the memory device or retrieved from an EHR change log database; in response to the EHR data differing from the prior version data, update the prior version data to match the EHR data or generate new prior version data to match the EHR data; receive first update data corresponding to an update to the EHR data, wherein the first update data is received from a first user device associated with a first user; in response to receiving the update data, generate notification data associated with a notification for a second user that the first user has proposed an update to the EHR data; transmit the notification data to a second user device associated with the second user; receive second update data from the second user device, wherein the second update data corresponds to the update to the EHR data; modify the prior version data or the new prior version data based on the first update data or the second update data; store the prior version data or the new prior version data on the memory device, or transmit the prior version data or the new prior version data to the EHR change log database; and transmit the first or second update data to the EHR database or modify the EHR data based on the first or second update data and transmit the EHR data as modified to the EHR database.

In some aspects, a device for EHR collaboration is described. The device may include computer-readable instructions that, when executed by a processor: receive EHR data, wherein the EHR data is indicative of an EHR; compare the EHR data to prior version data associated with the EHR data, wherein the prior version data is stored in the memory or retrieved from an EHR change log database; in response to the EHR data differing from the prior version data, update the prior version data to match the EHR data or generate new prior version data to match the EHR data; receive first update data corresponding to an update to the EHR data; in response to receiving the update data, generate notification data associated with a notification that an update to the EHR data has been proposed; transmit the notification data; receive second update data, wherein the second update data corresponds to the update to the EHR data; modify the prior version data or the new prior version data based on the update data; store the prior version data or the new prior version data in the memory, or transmit the prior version data or the new prior version data to the EHR change log database; and transmit the update data to an EHR database, or modify the EHR data based on the update data and transmit the EHR data as modified to the EHR database.

In some aspects, a method of EHR collaboration is described. The method may include: receiving EHR data from an EHR database, wherein the EHR data is indicative of an EHR; comparing the EHR data to prior version data associated with the EHR data, wherein the prior version data is stored on the memory device or retrieved from an EHR change log database; in response to the EHR data differing from the prior version data, updating the prior version data to match the EHR data or generate new prior version data to match the EHR data; receiving first update data corresponding to an update to the EHR data, wherein the first update data is received from a first user device associated with a first user; in response to receiving the update data, generating notification data associated with a notification for a second user that the first user has proposed an update to the EHR data; transmitting the notification data to a second user device associated with the second user; receiving second update data from the second user device, wherein the second update data corresponds to the update to the EHR data; modifying the prior version data or the new prior version data based on the first update data or the second update data; storing the prior version data or the new prior version data on the memory device, or transmitting the prior version data or the new prior version data to the EHR change log database; and transmitting the first or second update data to the EHR database or modifying the EHR data based on the first or second update data and transmit the EHR data as modified to the EHR database.

In some aspects, a system for EHR collaboration is described. The system may include: a roles module including a user database that correlates a user with a role; a workflow module including logic associated with a workflow by which EHR data is updated; and a review cycle module including logic associated with a review cycle by which an update to the EHR data via the workflow module is reviewed by two or more users having different roles, wherein the role corresponds to one or more of a permission to update the EHR data via the workflow module and a position of the user in the review cycle, and wherein at least a portion of the logic associated with the workflow is logically connected to the logic associated with the review cycle such that the update to the EHR data via the workflow triggers one or more actions in the review cycle.

In some aspects, a device for EHR collaboration is described. The device may have implemented thereon: a roles module that correlates a user indicated by a user database with a role indicated by the user database; a workflow module including logic associated with a workflow by which EHR data is updated; and a review cycle module including logic associated with a review cycle by which an update to the EHR data via the workflow module is reviewed by two or more users having different roles, wherein the role corresponds to one or more of a permission to update the EHR data via the workflow module and a position of the user in the review cycle, and wherein at least a portion of the logic associated with the workflow is logically connected to the logic associated with the review cycle such that the update to the EHR data via the workflow triggers one or more actions in the review cycle.

In some aspects, a method of EHR collaboration is described. The method may include: correlating a user with a role via a roles module that includes a user database, wherein the user database indicates the user and the role; receiving EHR data; updating the EHR data via a workflow module that includes logic associated with a workflow by which the EHR data is updated, wherein the role corresponds to a permission to update the EHR data via the workflow module; and triggering, in response to updating the EHR data via the workflow module, review logic associated with a review cycle module, wherein: an update to the EHR data via the workflow module is reviewed by two or more users having different roles; the role of the user corresponds to a position of the user in a review cycle indicated by the review logic; and at least a portion of the logic associated with the workflow is logically connected to the logic associated with the review cycle such that the update to the EHR data via the workflow triggers one or more actions in the review cycle.

In some aspects, a system for EHR collaboration is described. The system may include: an EHR data correlation module including comparison logic and an EHR change log database, wherein: EHR data is received by the EHR data correlation module from an EHR database, the EHR data indicative of an EHR; the EHR data is compared, by the EHR data correlation module, to prior version data associated with the EHR data, wherein the prior version data is stored in the EHR change log database; in response to the EHR data differing from the prior version data: the prior version data is updated by the EHR data correlation module to match the EHR data; or new prior version data is generated by the EHR data correlation module to match the EHR data; a roles module including a user database that correlates a user with a role; a workflow module including logic associated with a workflow by which EHR data is updated, wherein: first update data corresponding to an update to the EHR data is received via the workflow module from a first user associated with a first role indicated by the roles module; the first update data is handled via the logic associated with the workflow; and the workflow includes review trigger logic; and a review cycle module including logic associated with a review cycle by which the update to the EHR data via the workflow module is reviewed by one or more additional users, each of the one or more additional users having one or more different roles from the first user, wherein: the review trigger logic of the workflow module triggers notification logic of the review cycle by which a second user is notified of the update to the EHR data; and update logic of the review cycle module exports the update data to the EHR database or updates the EHR data according to the update data.

In some aspects, a device for EHR collaboration is described. The device may have implemented thereon: an EHR data correlation module including comparison logic, wherein: EHR data is received by the EHR data correlation module from an EHR database, the EHR data indicative of an EHR; the EHR data is compared, by the EHR data correlation module, to prior version data associated with the EHR data, wherein the prior version data is stored in an EHR change log database; in response to the EHR data differing from the prior version data: the prior version data is updated by the EHR data correlation module to match the EHR data; or new prior version data is generated by the EHR data correlation module to match the EHR data; a roles module that correlates a user indicated by a user database with a role indicated by the user database; a workflow module including logic associated with a workflow by which the EHR data is updated, wherein: update data corresponding to an update to the EHR data is received via the workflow module from a first user associated with a first role indicated by the roles module; the update data is handled via the logic associated with the workflow; and the workflow includes review trigger logic; and a review cycle module including logic associated with a review cycle by which the update to the EHR data via the workflow module is reviewed by one or more additional users, each of the one or more additional users having one or more different roles from the first user, wherein: the review trigger logic of the workflow module triggers notification logic of the review cycle by which a second user is notified of the update to the EHR data; and update logic of the review cycle module exports the update data to the EHR database or updates the EHR data according to the update data.

In some aspects, a method of EHR collaboration is described. The method may include: receiving, by an EHR data correlation module from an EHR database, EHR data indicative of an EHR; comparing, by the EHR data correlation module, the EHR data to prior version data associated with the EHR data, wherein the EHR data correlation module includes an EHR change log database in which the prior version data is stored; in response to the EHR data differing from the prior version data: updating, by the EHR data correlation module, the prior version data to match the EHR data, or generating, by the EHR data correlation module, new prior version data that matches the EHR data; correlating, by a roles module, a user with a role based using a user database of the roles module; receiving, via a workflow module, update data corresponding to an update to the EHR data, wherein the workflow module includes logic associated with a workflow by which the EHR data is updated, wherein the workflow module further includes review trigger logic; updating the EHR data by a workflow module based on the update data, wherein the update data is handled via the logic associated with the workflow; triggering, by the review trigger logic, notification logic of a review cycle module, wherein the review cycle module includes logic associated with a review cycle by which the update to the EHR data via the workflow module is reviewed by one or more additional users, each of the one or more additional users having one or more different roles from the first user, wherein the notification logic of the review cycle notifies a second user of the update to the EHR data; and exporting the update data to the EHR database or updating the EHR data according to the update data.

### BRIEF DESCRIPTION OF THE DRAWINGS

The present description will be understood more fully when viewed in conjunction with the accompanying drawings of various examples of devices, systems, and methods for EHR collaboration. The description is not meant to limit the devices, systems, and methods for EHR collaboration to the specific examples. Rather, the specific examples depicted and described are provided for explanation and understanding of devices, systems, and methods for EHR collaboration. Throughout the description the drawings may be referred to as drawings, figures, and/or FIGS.

FIG. 1 illustrates an EHR collaboration system, according to an implementation.

FIG. 2 illustrates a device schematic for various devices used in an EHR collaboration system, according to an implementation.

FIG. 3 illustrates a device schematic for a processing and/or memory device that may be used for EHR collaboration, according to an implementation.

FIG. 4 illustrates a device schematic for a processing and/or memory device with an EHR data correlation module, according to an implementation.

FIG. 5 illustrates a method of EHR collaboration by which an EHR is changed and/or updated, according to an implementation.

FIG. 6 illustrates a method of EHR collaboration via roles, workflow, and review cycle modules, according to an implementation.

FIG. 7 illustrates a method of EHR collaboration using an EHR data correlation module, according to an implementation.

FIGS. 8A-B illustrate a database schema for an EHR collaboration system, according to an implementation.

FIG. 9 illustrates various functions and nodes of a workflow module, according to an implementation.

FIG. 10 illustrates a user interface associated with the workflow module, according to an implementation.

FIG. 11 illustrates a review cycle in connection with various roles, according to an implementation.

FIG. 12 illustrates a change log associated with an EHR, according to an implementation.

FIG. 13 illustrates example data flow for update data, according to an implementation.

FIG. 14 illustrates example data flow that accounts for previously-denied changes, according to an implementation.

### DETAILED DESCRIPTION

Devices, systems, and methods for EHR collaboration as disclosed herein will become better understood through a review of the following detailed description in conjunction with the figures. The detailed description and figures provide merely examples of the various implementations of devices, systems, and methods for EHR collaboration. Many variations are contemplated for different applications and design considerations; however, for the sake of brevity and clarity, all the contemplated variations may not be individually described in the following detailed description. Those skilled in the art will understand how the disclosed examples may be varied, modified, and altered and not depart in substance from the scope of the examples described herein.

Conventional EHRs are stored in databases owned by a company that manages the EHR. EHR stakeholders may vary between healthcare systems, but may generally include clinicians (e.g., nurses, doctors, pharmacists, and so forth), clinical leaders such as managers, technicians, and business operations staff (e.g., in supply chain, finance, legal, logistics, and so forth). Stakeholders for a particular EHR may be employed by different organizations, or different departments in the same organization. Those stakeholders may engage in modifying data elements of the EHR to support their care needs and variations. For example, an EHR data element may be for a particular medication. That EHR data element may offer guidance via, e.g., a dose button. Stakeholders impacted by adjustments to the medication data in the EHR may include doctors, nurses and/or medical assistants that support doctors, administrative staff for the doctor's office/practice, pharmacist staff, the pharmacy technician that works with the pharmacist, operations personnel that coordinate delivery of the medication to the pharmacy and/or to the patient, technology personnel developing and/or managing automation, personnel of ambulatory practice groups, personnel associated with niche groups such as opioid steering groups or smart pump infusion oversight, and so forth.

Stakeholders may need access to the EHR and/or other EHR data elements such as the medication data. In some cases, a stakeholder may need to update the EHR based, for example, on an updated protocol, dosage, or newly identified best practice. An update may impact other processes or features of an EHR. Multiple people and/or departments within an organization may and across different organizations may be involved in updating an EHR. In general, ensuring all stakeholders are aware of and contribute as needed to updating and EHR is a challenging, linear or non-linear process. For example, a process may include a ticketing system, which may be organized by a spreadsheet or some other internal database system for an organization. An individual may be responsible for ensuring that all parties necessary for a change to an EHR are notified. This, however, does not facilitate coordination with other organizations. Indeed, most systems for handling EHR updates are ad hoc, with little to no coordination between different organizations. This leads to delays, information being missed or omitted, and makes it nearly impossible for different health systems to communicate with each other regarding best practices in an agile manner.

Implementations of the devices, systems, and methods for EHR collaboration may address some or all of the problems described above. Generally, the devices, systems, and methods collectivize EHR changes by extracting EHR data, creating a change log for a particular EHR, and then publishing the changes to the EHR once the change protocol has been completed. A change to an EHR may be accomplished using a change package, which may be standardized for a particular type of EHR data (e.g., medication data, surgical data, lab data, and so forth). The change package may vary depending on the type of user that initiates the change package (e.g., a provider, a pharmacist, and so forth). Users across different organizations may have different roles in the change package. The devices, systems, and methods described herein may provide for notification of users regarding a pending EHR change and their role in the change package. Linear and non-linear change packages may be implemented.

Changes made to an EHR are stored for use by other parties that do not participate in the change package. For example, a health system may implement a new process for dispensing a particular medication based on clinical observations. Users of other health systems may be notified of the change, such as when updating their EHR for the medication. This may allow for communication of best practices across different health systems.

The devices, systems, and methods for EHR collaboration described herein address several problems with current methodologies for handling EHRs. For example, EHR collaboration as described herein allows for multiple organizations to coordinate on changes to an EHR. EHR collaboration as described herein may allow for a standardized record of changes so that the evolution of an EHR is recorded. EHR collaboration as described herein may allow for communication of best practices across different health systems.

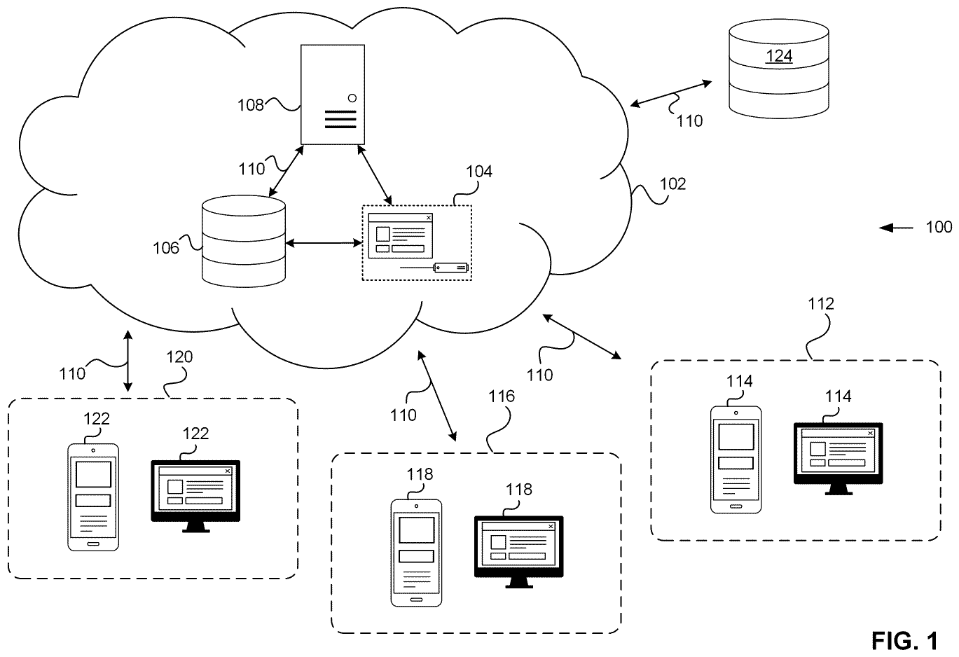

FIG. 1 illustrates an EHR collaboration system 100, according to an implementation. In various implementations, the EHR collaboration system 100 may include a cloud-based data management system 102. In some implementations, the cloud-based data management system 102 may include an application server 104 through which remote devices may communicate with the cloud-based data management system 102. The cloud-based data management system 102 may include a memory device 106 and a processing device 108. The cloud-based data management system 102 may include one or more communication devices that form communication links 110 between the various components of the cloud-based data management system 102 and/or between the cloud-based data management system 102 and various external devices, e.g., user devices, such as described below.

In various implementations, the EHR collaboration system 100 may include a first user set 112 that may include one or more first user devices 114, a second user set 116 that may include one or more second user devices 118, and a third user set 120 that may include third user devices 122. The user devices of the user sets may communicate with the cloud-based data management system 102 via one or more of the communication links 110. The user devices may communicate with each other one or more of the communication links 110.

The user sets may correlate with different groups of users of the EHR collaboration system 100, such as different groups of stakeholders of a health system. A user set may include one or more users corresponding to the one or more user devices of the set. For example, the first user set 112 may include two instances of the first user devices 114, which in turn may correspond to two different users who are part of the first user set 112. More specifically, the first user set 112 may correspond to a particular role in the EHR collaboration system 100, such as a change approval role, or a change review role. The second user set 116 and the third user set 120 may correspond to different roles. In some implementations, user devices within the same user set may correspond to users with different roles. For example, the first user set 112 may correspond to a pharmacy group. A user within the first user set 112 may be a pharmacist with an approval role. A user within the first user set 112 may be a pharmacy technician with a review role.

The EHR collaboration system 100 may include an EHR database 124. The EHR database 124 may store EHRs. The structure of the EHR database 124 may depend on the vendor that provides the EHR database 124. In some implementations, the EHR database 124 may be a relational database. In some implementations, the EHR database 124 may be a hierarchical database. The EHR database 124 may be structured or unstructured. The EHR database 124 may communicate with the cloud-based data management system 102 and/or one or more of the user devices via one or more of the communication links 110.

The EHR collaboration system 100 may be web-based. A user device (e.g., user devices 114, 118, and/or 122) may access the cloud-based data management system 102 via an online portal. The online portal may be served to the user devices via the application server 104.

Instructions for implementing the application server 104 may be stored in the memory device 106 and/or on the processing device 108. The instructions may be implemented by the processing device 108, or by a separate processing device dedicated to the application server 104.

The EHR collaboration system 100 may be implemented using a public internet. The EHR collaboration system 100 may be implemented using a private intranet. Elements of the cloud-based data management system 102, such as the application server 104, the memory device 106, the processing device 108, and/or the communication links 110, may be physically housed at a location remote from an entity that owns and/or operates the EHR collaboration system 100. For example, various elements of the EHR collaboration system 100 may be physically housed at a public service provider such as a web services provider. Elements of the EHR collaboration system 100 may be physically housed at a private location, such as at a location occupied by the entity that owns and/or operates the EHR collaboration system 100.

The communication links 110 may be direct or indirect. A direct link may include a link between two devices where information is communicated from one device to the other without passing through an intermediary. For example, the direct link may include a Bluetooth™ connection, a Zigbee® connection, a Wifi Direct™ connection, a near-field communications (NFC) connection, an infrared connection, a wired universal serial bus (USB) connection, an ethernet cable connection, a fiber-optic connection, a firewire connection, a microwire connection, and so forth. In another example, the direct link may include a system bus, a control bus, a data bus, and address bus, a cable on a bus network, and so forth.

An indirect link may include a link between two or more devices where data may pass through an intermediary, such as a router, before being received by an intended recipient of the data. For example, the indirect link may include a wireless fidelity (WiFi) connection where data is passed through a WiFi router, a cellular network connection where data is passed through a cellular network router, a wired network connection where devices are interconnected through hubs and/or routers, and so forth. The cellular network connection may be implemented according to one or more cellular network standards, including the global system for mobile communications (GSM) standard, a code division multiple access (CDMA) standard such as the universal mobile telecommunications standard, an orthogonal frequency division multiple access (OFDMA) standard such as the long-term evolution (LTE) standard, and so forth.

Various of the elements of the EHR collaboration system 100 may include data storage and/or processing capabilities (e.g., the application server 104, the memory device 106, the processing device 108, the first user devices 114, the second user devices 118, the third user devices 122, and/or the EHR database 124). Such capabilities may be rendered by various electronics for processing and/or storing electronic signals. One or more of the devices in the EHR collaboration system 100 may include a processing device. For example, the cloud-based application server 104, the memory device 106, the processing device 108, the first user devices 114, the second user devices 118, the third user devices 122, and/or the EHR database 124 may include a processing device. One or more of the devices in the EHR collaboration system 100 may include a memory device. For example, the cloud-based application server 104, the memory device 106, the processing device 108, the first user devices 114, the second user devices 118, the third user devices 122, and/or the EHR database 124 may include the memory device. The processing device may have volatile and/or persistent memory. The memory device may have volatile and/or persistent memory. The processing device may have volatile memory and the memory device may have persistent memory.

The processing device may generate an output based on an input. For example, the processing device may receive an electronic and/or digital signal. The processing device may read the signal and perform one or more tasks with the signal, such as performing various functions with data in response to input received by the processing device. The processing device may read from the memory device information needed to perform the functions. For example, the processing device may update a variable from static to dynamic based on a received input and a rule stored as data on the memory device. The processing device may send an output signal to the memory device, and the memory device may store data according to the signal output by the processing device.

The processing device may be and/or include a processor, a microprocessor, a computer processing unit (CPU), a graphics processing unit (GPU), a neural processing unit, a physics processing unit, a digital signal processor, an image signal processor, a synergistic processing element, a field-programmable gate array (FPGA), a sound chip, a multi-core processor, and so forth. As used herein, “processor,” “processing component,” “processing device,” and/or “processing unit” may be used generically to refer to any or all of the aforementioned specific devices, elements, and/or features of the processing device.

The memory device may be and/or include a computer processing unit register, a cache memory, a magnetic disk, an optical disk, a solid-state drive, and so forth. The memory device may be configured with random access memory (RAM), read-only memory (ROM), static RAM, dynamic RAM, masked ROM, programmable ROM, erasable and programmable ROM, electrically erasable and programmable ROM, and so forth. As used herein, “memory,” “memory component,” “memory device,” and/or “memory unit” may be used generically to refer to any or all of the aforementioned specific devices, elements, and/or features of the memory device.

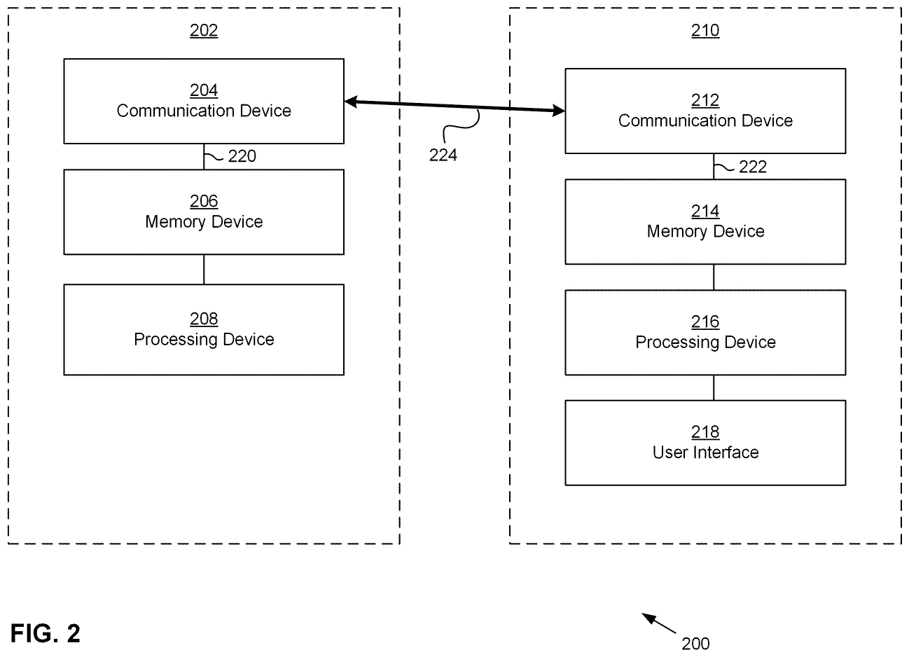

Various of the devices in the EHR collaboration system 100 may include data communication capabilities. Such capabilities may be rendered by various electronics for transmitting and/or receiving electronic and/or electromagnetic signals. One or more of the devices in the EHR collaboration system 100 may include a communication device, e.g., those communication devices described regarding FIG. 2. For example, the cloud-based application server 104, the memory device 106, the processing device 108, the first user devices 114, the second user devices 118, the third user devices 122, and/or the EHR database 124 may include a communication device.

The communication device may include, for example, a networking chip, one or more antennas, and/or one or more communication ports. The communication device may generate radio frequency (RF) signals and transmit the RF signals via one or more of the antennas. The communication device may receive and/or translate the RF signals. The communication device may transceive the RF signals. The RF signals may be broadcast and/or received by the antennas.

The communication device may generate electronic signals and transmit the RF signals via one or more of the communication ports. The communication device may receive the RF signals from one or more of the communication ports. The electronic signals may be transmitted to and/or from a communication hardline by the communication ports. The communication device may generate optical signals and transmit the optical signals to one or more of the communication ports. The communication device may receive the optical signals and/or may generate one or more digital signals based on the optical signals. The optical signals may be transmitted to and/or received from a communication hardline by the communication port, and/or the optical signals may be transmitted and/or received across open space by the networking device.

The communication device may include hardware and/or software for generating and communicating signals over a direct and/or indirect network communication link. For example, the communication component may include a USB port and a USB wire, and/or an RF antenna with Bluetooth™ programming installed on a processor, such as the processing component, coupled to the antenna. In another example, the communication component may include an RF antenna and programming installed on a processor, such as the processing device, for communicating over a Wifi and/or cellular network. As used herein, “communication device” “communication component,” and/or “communication unit” may be used generically herein to refer to any or all of the aforementioned elements and/or features regarding communication.

Various of the elements in the EHR collaboration system 100 may be referred to as a “server.” Such elements may include a server device. The server device may include a physical server and/or a virtual server. For example, the server device may include one or more bare-metal servers. The bare-metal servers may be single-tenant servers or multiple tenant servers. In another example, the server device may include a bare metal server partitioned into two or more virtual servers. The virtual servers may include separate operating systems and/or applications from each other. In yet another example, the server device may include a virtual server distributed on a cluster of networked physical servers. The virtual servers may include an operating system and/or one or more applications installed on the virtual server and distributed across the cluster of networked physical servers. In yet another example, the server device may include more than one virtual server distributed across a cluster of networked physical servers.

The term server may refer to functionality of a device and/or an application operating on a device. For example, an application server may be programming instantiated in an operating system installed on a memory device and run by a processing device. The application server may include instructions for receiving, retrieving, storing, outputting, and/or processing data. A processing server may be programming instantiated in an operating system that receives data, applies rules to data, makes inferences about the data, and so forth. Servers referred to separately herein, such as an application server, a processing server, a collaboration server, a scheduling server, and so forth may be instantiated in the same operating system and/or on the same server device. Separate servers may be instantiated in the same application or in different applications.

Various aspects of the systems described herein may be referred to as “data.” Data may be used to refer generically to modes of storing and/or conveying information. Accordingly, data may refer to textual entries in a table of a database. Data may refer to alphanumeric characters stored in a database. Data may refer to machine-readable code. Data may refer to images. Data may refer to audio. Data may refer to, more broadly, a sequence of one or more symbols. The symbols may be binary. Data may refer to a machine state that is computer-readable. Data may refer to human-readable text.

Various of the devices in the EHR collaboration system 100 may include a user interface for outputting information in a format perceptible by a user and receiving input from the user, e.g., as shown and described regarding FIG. 2. The user interface may include a display screen such as a light-emitting diode (LED) display, an organic LED (OLED) display, an active-matrix OLED (AMOLED) display, a liquid crystal display (LCD), a thin-film transistor (TFT) LCD, a plasma display, a quantum dot (QLED) display, and so forth. The user interface may include an acoustic element such as a speaker, a microphone, and so forth. The user interface may include a button, a switch, a keyboard, a touch-sensitive surface, a touchscreen, a camera, a fingerprint scanner, and so forth. The touchscreen may include a resistive touchscreen, a capacitive touchscreen, and so forth.

FIG. 2 illustrates a device schematic 200 for various devices used in the EHR collaboration system 100, according to an implementation. A server device 202 may include a communication device 204, a memory device 206, and a processing device 208. The server device 202 may, for example, be implemented in the EHR collaboration system 100 as the application server 104, the memory device 106, the processing device 108, and/or the EHR database 124. A client device 210 may include a communication device 212, a memory device 214, a processing device 216, and a user interface 218. The client device 210 may be implemented in the EHR collaboration system 100 as, for example, one or more of the first user devices 114, the second user devices 118, and/or the third user devices 122. Components of the server device 202 may communicate via communication links 220. Components of the client device 210 may similarly communicate with each other via communication links 222. The server device 202 may communicate with the client device 210 via an external communication link 224, such as between the communication device 204 and the communication device 212.

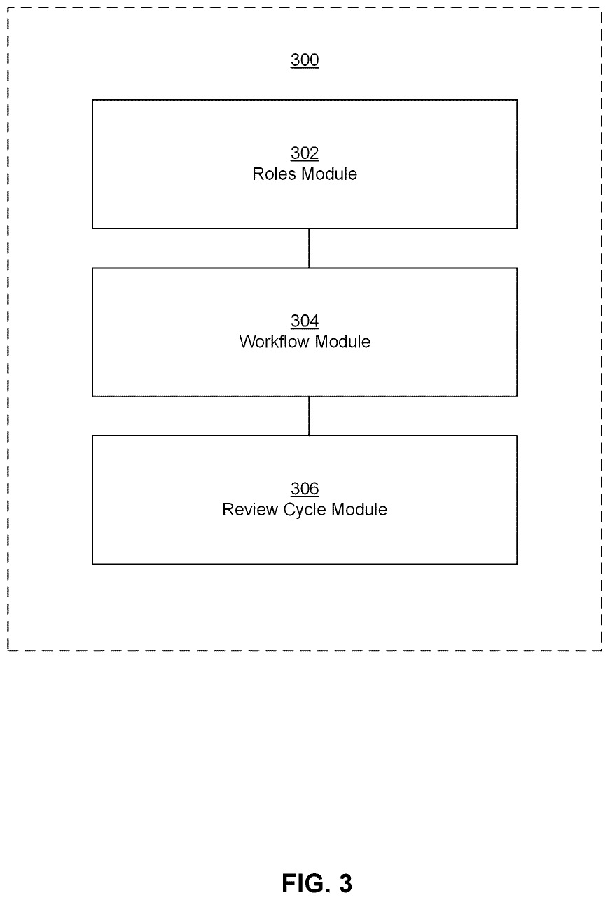

FIG. 3 illustrates a device schematic 300 for a processing and/or memory device that may be used for EHR collaboration, e.g., as a stand-alone device (in connection with other conventional device components such as a communication device and/or user interface) or in the EHR collaboration system 100, according to an implementation. The processing and/or memory device may include a roles module 302, a workflow module 304, and/or a review cycle module 306. The roles module 302 may include a user database that correlates a user with a role. The role may correspond to one or more of a permission to update EHR data via the workflow module 304 and a position of the user in the review cycle. The position of the user in the review cycle may be indicated by the review cycle module 306. The workflow module 304 may include logic associated with a workflow by which EHR data is updated. The review cycle module 306 may include logic associated with a review cycle by which an update to the EHR data via the workflow module 304 is reviewed by two or more users having different roles. The roles may be specified in the roles module 302. At least a portion of the logic associated with the workflow may be logically connected to the logic associated with the review cycle. An update to the EHR data via the workflow may trigger one or more actions in the review cycle.

As an example, a server may store and/or execute computer-readable instructions associated with one or more of the roles module 302, the workflow module 304, and the review cycle module 306. In the roles module 302, a user may be assigned one or more roles, e.g., an updating role that grants permission for the user to change an EHR and a review role that requires the user to review particular changes to particular types of EHRs that the user did not initiate. The roles may be associated with and/or leveraged by logic of the workflow module 304 and/or the review cycle module 306. For example, the workflow module 304 may limit which aspects of an EHR the user can change based on the role and/or permissions assigned in the roles module 302. As another example, the review cycle module 306 may include logic that notifies the user when a change is made to an EHR based on the roles module 302 indicating the user has a review role for the change.

As a more specific example, the roles module 302 may indicate a user is a doctor associated with an opioid steering committee. The roles module 302 may further indicate that the doctor has change permissions for opioid-related EHRs. The roles module 302 may further indicate that the doctor is a required reviewer for specific types of changes to opioid-related EHRs, such as dosage, prescription renewal periods, and so forth. When the doctor-user initiates a change to an opioid-related EHR, the workflow module 304 may prompt the user for certain information, restrict what aspects of the EHR the user may change, make recommendations based on similar changes made by other users to similar EHRs, and so forth. The workflow module 304 may also trigger the review cycle module 306 when the change to the EHR is submitted. When another user submits a change to the EHR, the review cycle module 306 may include notifying the doctor-user of the proposed change. The review cycle module 306 may require some action by the doctor-user, e.g., approval of a change, review of other aspects of the EHR to ensure viability of the change, marking a change as provisional pending additional clinical evidence, and so forth.

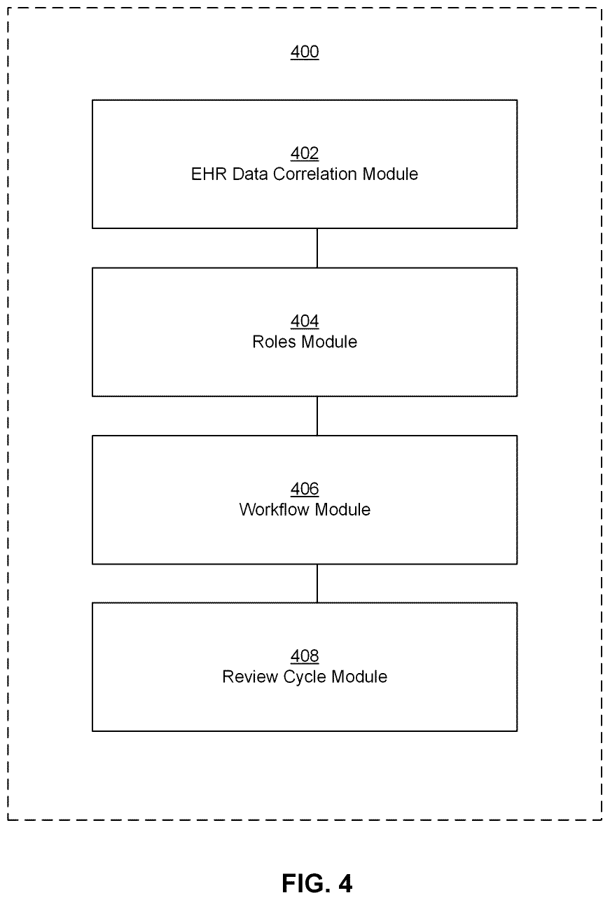

FIG. 4 illustrates a device schematic 400 for a processing and/or memory device with an EHR data correlation module, according to an implementation. The processing and/or memory device may be used for EHR collaboration, e.g., as a standalone device (in connection with other conventional device components such as a communication device and/or user interface) or in the EHR collaboration system 100. The device may include an EHR data correlation module 402, a roles module 404, a workflow module 406, and/or a review cycle module 408. Similar to that described above regarding FIG. 3, the roles module 404 may include a user database that correlates a user with a role, the workflow module 406 my include logic associated with a workflow by which EHR data is updated and/or changed and/or by which a review cycle is triggered, and the review cycle module 408 may include logic associated with the review cycle by which the update and/or change to the EHR data via the workflow module 406 is reviewed by one or more users. The one or more users may have different roles from a user that implements the change.

The EHR data correlation module 402 may include comparison logic and/or an EHR change log database. The comparison logic may compare EHR data associated with a particular EHR to change log data associated with the EHR. The change log data may indicate a prior version of the EHR, such as by including prior version data associated with the EHR. The EHR data correlation module 402 may include logic for updating prior version data associated with the EHR and/or generating new prior version data. Such logic may be triggered when, for example, the EHR data differs from the change log data and/or the prior version data. In various implementations, the logic of the EHR data correlation module 402 may assume that the EHR data is correct and represents the most up-to-date version of the EHR. In some implementations, the EHR data correlation module 402 may include logic that ensures the EHR data represents the most up-to-date version of the EHR, such as by checking metadata associated with the EHR data that indicates, for example, the date associated with the version of the EHR. If the metadata indicates the EHR version is newer than the version indicated by, e.g., the prior version data, the EHR data correlation module 402 may assume the new EHR data represents the most up-to-date version. If the metadata indicates the EHR version is older than the version indicated by, e.g., the prior version data, the EHR data correlation module 402 may assume the prior version data represents the most up-to-date version of the EHR.

Various methods are described below. The methods may be implemented by the EHR collaboration system 100, various elements of the EHR collaboration system 100 described above, the system described regarding FIG. 2, and/or one or more of the devices described regarding FIGS. 3 and/or 4. For example, inputs indicated as being received in a method may be input at the first user devices 114, the second user devices 118, and/or the third user devices 122 and/or received at the cloud-based data management system 102. Comparisons, updates, modifications, and/or correlations performed in the methods may be associated with instructions stored in a memory device and/or executed by a processing device. Data generated in a method may be outputs generated by a processing device, stored by a memory device, and/or communicated by a communication device. Data transmitted or exported in a method may be done so by a communication device. Triggering logic and/or instructions may be processed by a processing device. In general, data described in the methods may be stored and/or processed by various elements of the EHR collaboration system 100, the system described regarding FIG. 2, and/or the devices described regarding FIGS. 3 and/or 4.

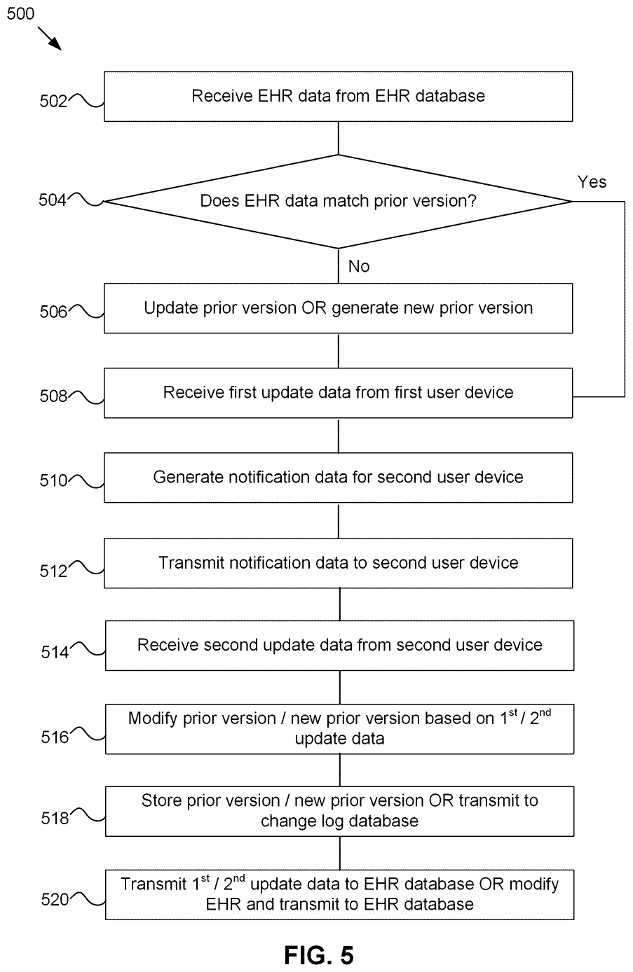

FIG. 5 illustrates a method 500 of EHR collaboration by which an EHR is changed and/or updated, according to an implementation. The method 500 may include receiving EHR data from an EHR database (block 502). The EHR data may be indicative of an EHR. The method 500 may include comparing the EHR data to prior version data associated with the EHR data (block 504). The prior version data may be stored on a memory device. The prior version data may be retrieved from an EHR change log database. The method 500 may include, in response to the EHR data differing from the prior version data, updating the prior version data to match the EHR data or generating new prior version data to match the EHR data (block 506).

The method 500 may include receiving first update data corresponding to an update to the EHR data (block 508). The first update data may be received from a first user device associated with a first user. The method 500 may include, in response to receiving the update data, generating notification data associated with a notification for a second user that the first user has proposed an update to the EHR data (block 510). The method 500 may include transmitting the notification data to a second user device associated with the second user (block 512).

The method 500 may include receiving second update data from the second user device (block 514). The second update data may correspond to the update to the EHR data. The method 500 may include modifying the prior version data or the new prior version data based on the first update data or the second update data (block 516). The method 500 may include storing the prior version data or the new prior version data on the memory device or transmitting the prior version data or the new prior version data to the EHR change log database (block 518). The method 500 may include transmitting the first or second update data to the EHR database or modifying the EHR data based on the first or second update data and transmitting the EHR data as modified to the EHR database (block 520).

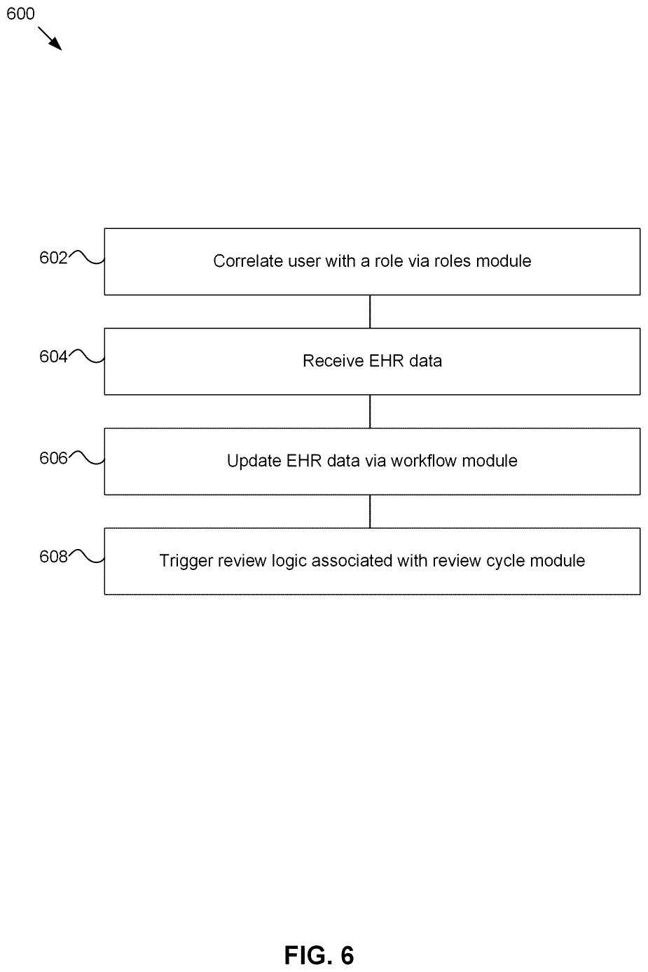

FIG. 6 illustrates a method 600 of EHR collaboration via roles, workflow, and review cycle modules, according to an implementation. The method 600 may include correlating a user with a role via a roles module (block 602). The roles module may include a user database. The user database may indicate the user and the role. The method 600 may include receiving EHR data (block 604). The method 600 may include updating the EHR data via a workflow module (block 606). The workflow module may include logic associated with a workflow. The EHR data is updated via such logic. The role indicated in the roles module may correspond to a permission to update the EHR data via the workflow module. The method 600 may include triggering review logic associated with a review cycle module (block 608). Such logic may be triggered in response to updating the EHR data via the workflow module.

An update to the EHR data via the workflow module may be reviewed by two or more users having different roles. The roles of the respective users may correspond to a position of the user in a review cycle indicated by the review logic. At least a portion of the logic associated with the workflow may be logically connected to the logic associated with the review cycle such that the update to the EHR data via the workflow triggers one or more actions in the review cycle.

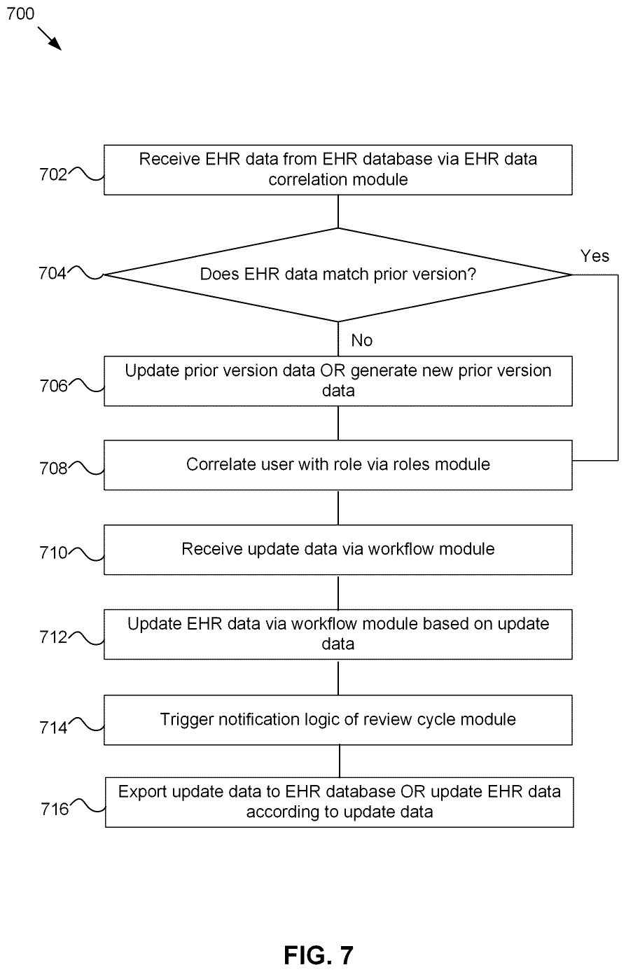

FIG. 7 illustrates a method 700 of EHR collaboration using an EHR data correlation module, according to an implementation. The method 700 may include receiving EHR data indicative of an EHR (block 702). The EHR data may be received by an EHR data correlation module. The EHR data may be received from an EHR database. The method 700 may include comparing the EHR data to prior version data associated with the EHR data (block 704). The comparison may be performed by the EHR data correlation module. The EHR data correlation module may include an EHR change log database in which the prior version data is stored. The method 700 may include updating the prior version data to match the EHR data or generating new prior version data that matches the EHR data (block 706). The updating or generating may be performed by the EHR data correlation module. The updating or generating may be in response to the EHR data differing from the prior version data.

The method 700 may include correlating a user with a role (block 708). The correlation may be performed by a roles module. The roles module may include and/or utilize a user database. The correlation may be based on the user database. The method 700 may include receiving update data corresponding to an update to the EHR data (block 710). The update data may be received via a workflow module. The workflow module may include logic associated with a workflow by which the EHR data is updated. The workflow module may include review trigger logic. The method 700 may include updating the EHR data by the workflow module based on the update data (block 712). The update data may be handled via the logic associated with the workflow.

The method 700 may include triggering, by the review trigger logic, notification logic of a review cycle module (block 714). The review cycle module may include logic associated with a review cycle by which the update to the EHR data via the workflow module is reviewed by one or more additional users. The one or more additional users may have one or more different roles from the first user. The notification logic of the review cycle may notify a second user of the update to the EHR data. The method 700 may include exporting the update data to the EHR database or updating the EHR data according to the update data (block 716).

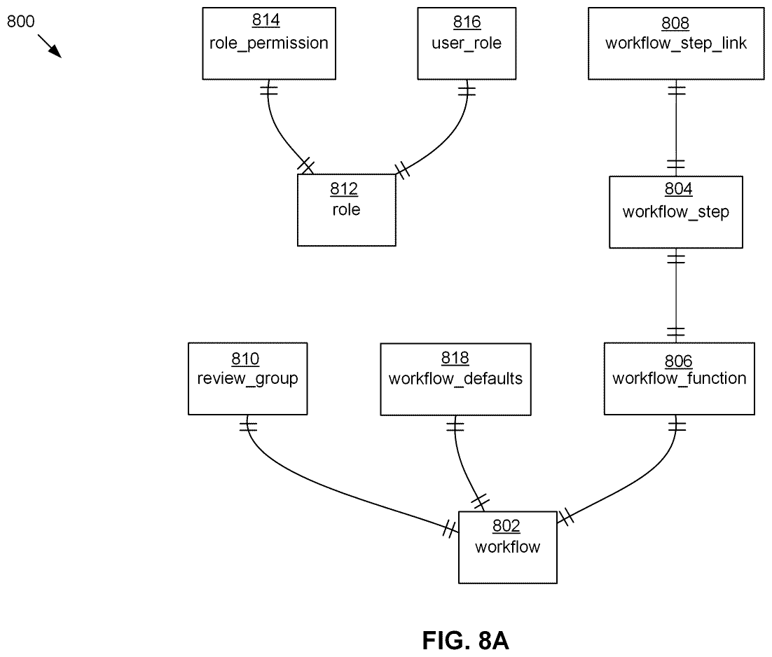

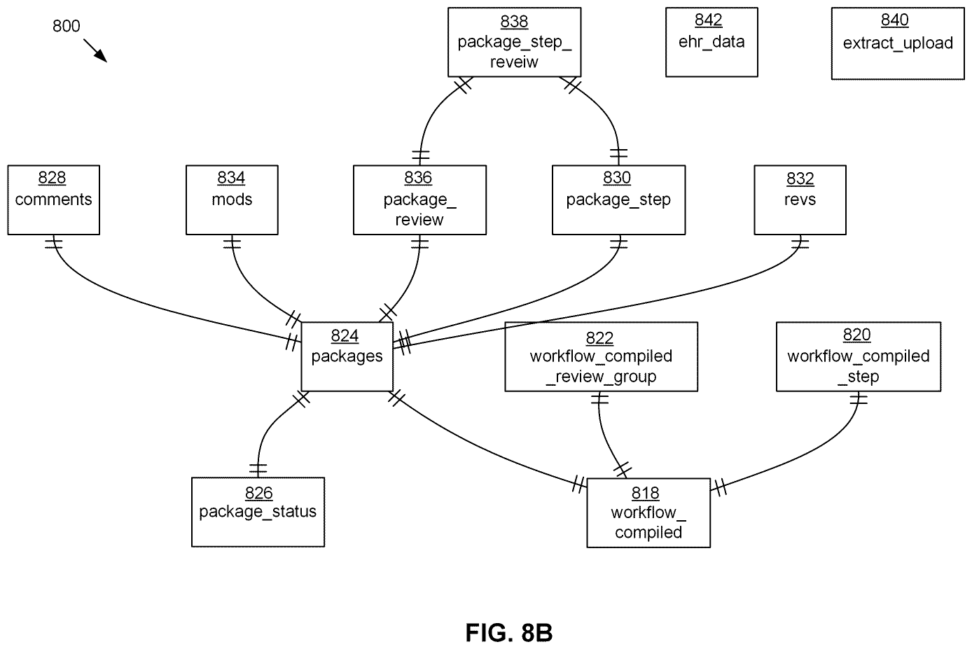

FIGS. 8A-B illustrate a database schema 800 for an EHR collaboration system, according to an implementation. The database design is illustrative of an implementation of the designs disclosed above. As shown, workflows 802 may be formed from tables of steps 804, functions 806, and/or links 808 between steps. Workflows may be linked to review groups 810 that may be informed by roles 812 having connections to role permissions 814 and users 816. Certain defaults 818 may be stored for particular workflows. This set of data may be compiled within a change package 818 to inform its structure 820 and review 822 expectation. Change packages are then stored via tables 824 that manage their status 826, comments 828, steps 830, a global revision number 832 that may be used for history tracking, modifications 834, review history 836, and step review history 838. EHR data may be exchanged 840 with an external EHR vendor and stored 842 in a database for adjustment and/or updating.

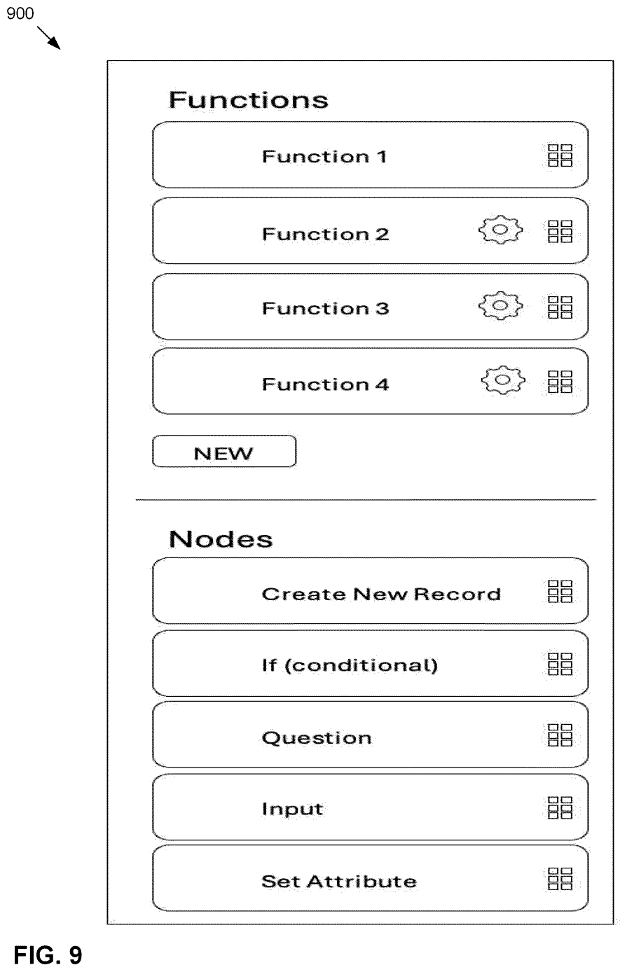

FIG. 9 illustrates various functions and nodes 900 of a workflow module, according to an implementation. Functions describe provisioned user-set rules/instructions for processing change-related data. Nodes are processing instructions that a provisioned user would use to populate the functions to create the rules. For example, an institution may wish to offer a mechanism to suggest a medication is refrigerated as a change. A function may be created to describe institutional rules and manners in which questions are asked on whether a medication should be refrigerated. This may include using a ‘Question’ node to present a question to end users on if something should be refrigerated. The other nodes may allow for that question to be processed into discrete changes to the EHR data that the change may impact, such as medication preparation instructions, within the logic of the system. Functions may include institutional rules and/or logic that is customizable by a user. The nodes may be tools for populating the function.

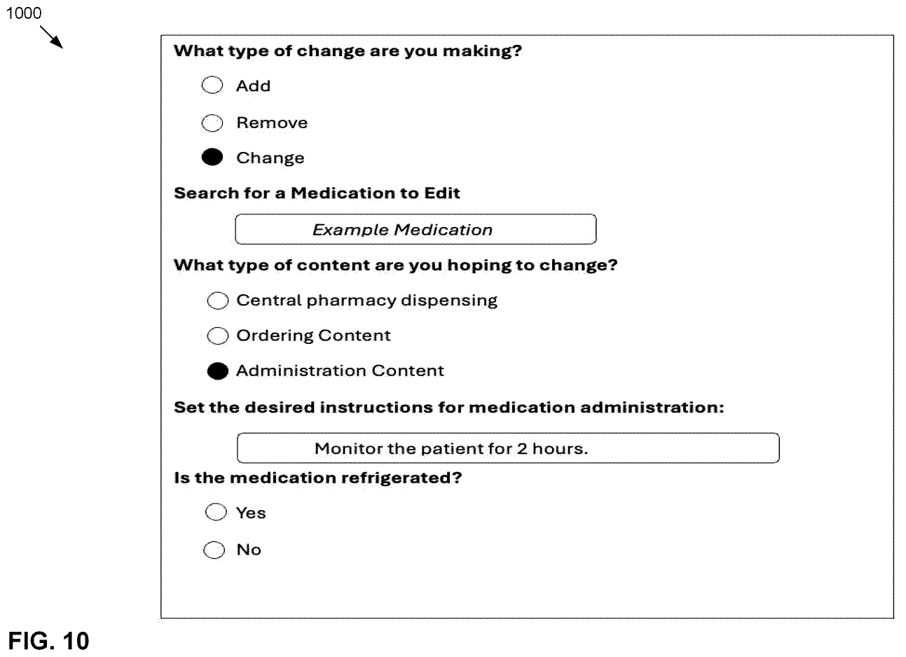

FIG. 10 illustrates a user interface 1000 associated with the workflow module, according to an implementation. A workflow may be populated with a series of questions and/or other user interface elements by which a user may suggest modifications to EHR data. For example, a change to a medication may be suggested in which discrete EHR data may be editable via direct adjustments and/or via answering questions that are then processed via one or more functions. A user may leverage this interface to suggest changes. The information displayed in the interface shown may be changed, e.g., by a user with a role that has such permissions, using the interface, nodes, and/or functions shown in FIG. 9.

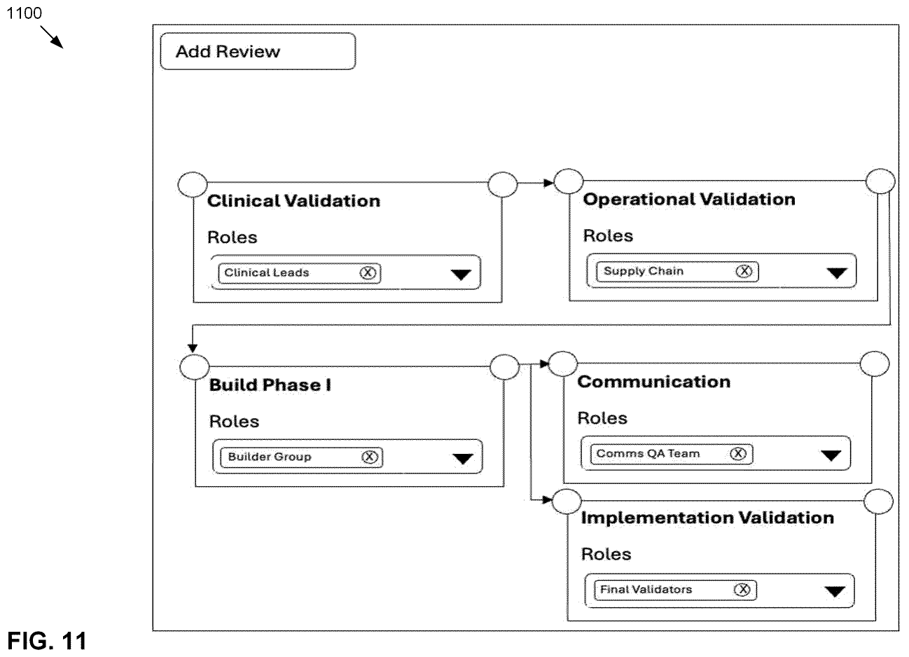

FIG. 11 illustrates a review cycle 1100 in connection with various roles, according to an implementation. A user may be provisioned with one or more roles that grant them access to aspects of the system such as workflows or other administrative functions. Review steps may be created to form a review cycle that may include one or more institutionally described roles. These review steps may be mapped in a linear or non-linear manner by which the institution may review suggested changes. For example, assessment of which products an institution stocks may be an early step in review. This step in the review cycle may include, for example, review by various operational leaders, logistic and supply chain staff, and so forth. Specific questions and/or content from workflows may be tagged for review. Group review of a change package may occur linearly or non-linearly (e.g., simultaneous review by various users with different roles). Notifications may be sent to user devices associated with users having roles pertinent to a change package. Content may be adjusted, approved, and/or denied, such as by the method 500 disclosed above. By use of this mapping, users are notified to review a change when appropriate for a particular change package. In some instances, user notifications may be limited to users for which content pertinent to their review is contained with the change package.

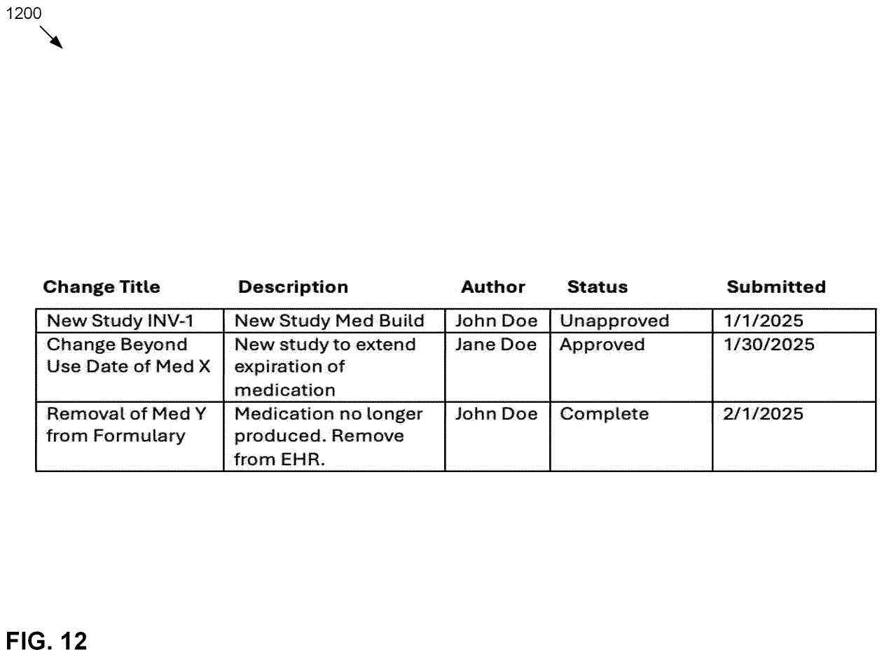

FIG. 12 illustrates a change log interface 1200 associated with an EHR, according to an implementation. A change suggestion may be contained within a change package. The change log interface 1200 may include a status indicator that indicates a progress of the change package through a review cycle. This “dashboard” may include metrics pertinent to oversight of various change packages. Using the change log interface 1200, a user may track the status and/or history of changes made. The change log interface 1200 may include summary content that summarizes multiple and/or all change packages submitted. The change log interface 1200 may include summary content that summarizes multiple and/or all change packages on the basis of data elements being adjusted with link-back to the change package that manipulated it.

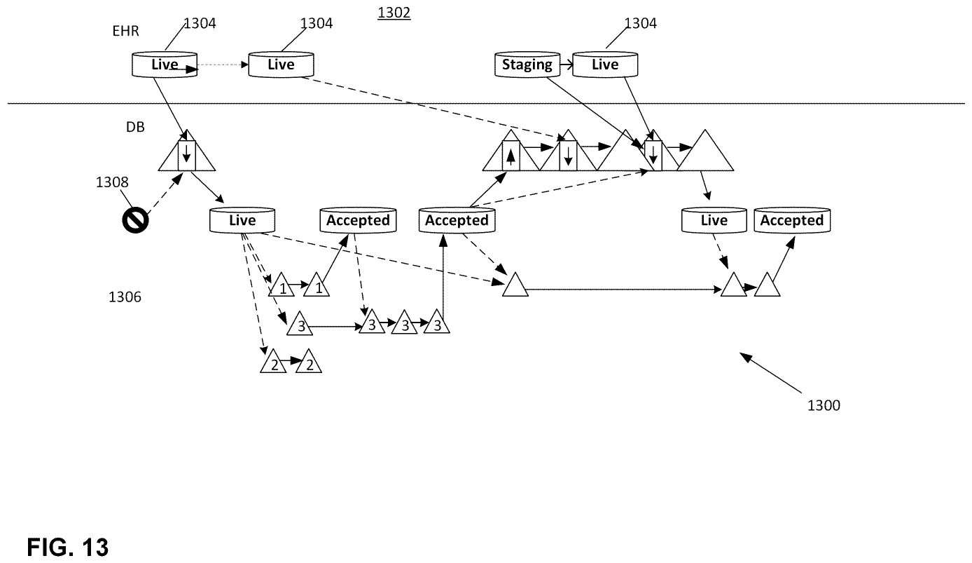

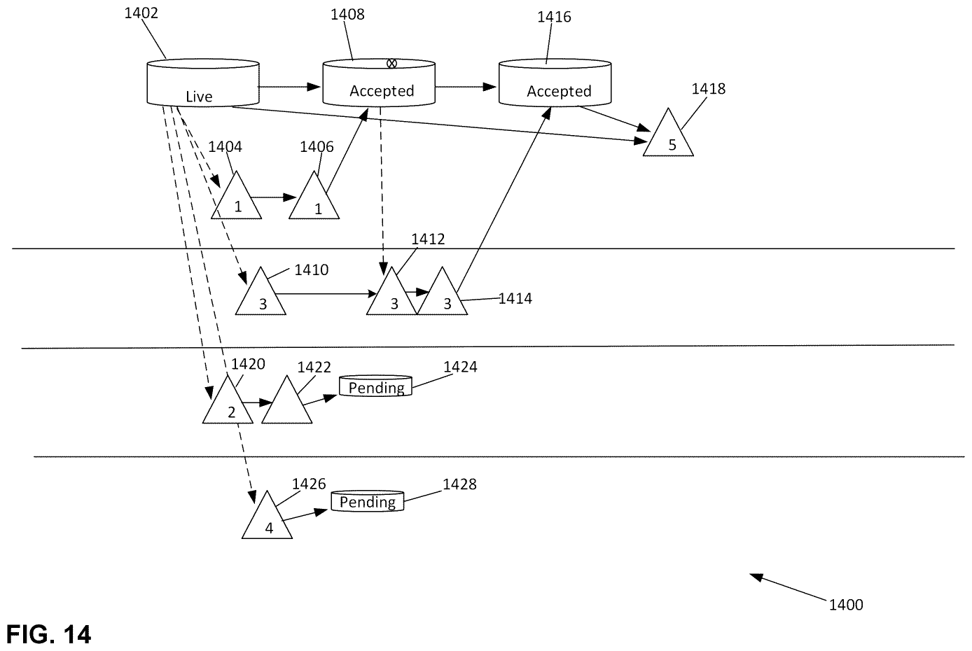

FIG. 13 illustrates example data flow for update data, according to an implementation. An EHR database 1302 may host live EHR data 1304. The live EHR data 1304 may be passed into a collaboration database 1306 with error checking 1308. The error checking may check the EHR data 1304 to ensure it is in an expected format and/or design. If the error checking 1308 fails, the EHR data 1304 will not be downloaded to the collaboration database 1306. The depicted data flow positions the collaboration system to model the EHR data 1304 for change suggestion. Multiple changes may occur to the EHR data 1304, and the changes may go through review processes prior to being fully approved/accepted. The change packages will then be uploaded back to the EHR database 1302 which should then reflect and mirror further EHR-retrieved data. If data conflicts, based on implementation, data will be selected to reflect the currently-live state for change packages. If changes are pending that are based on prior-live data, further review to ensure data integrity may occur. Data handling with respect to changes in the collaboration database 1306 is depicted in further detail with regard to FIG. 14.

FIG. 14 illustrates example data flow 1400 that accounts for situations in which the same data is being manipulated across multiple change packages, according to various implementations. In some implementations, data 1402 may be adjusted 1404, approved 1406, and applied 1408. In some implementations, the same data element may be adjusted again 1410. In some such implementations, the second adjustment 1410 does not conflict with the prior adjustment 1404, and may be assumed to be appropriate, i.e., additional review may not be implemented. The first adjustment 1404 and the second adjustment 1410 may be combined 1412, approved 1414, and applied 1416. In general, multiple adjustments 1418 may occur simultaneously, e.g., adjustments to the same EHR or data element by different stakeholders.

In some implementations, an adjustment 1420 may conflict 1422 with other adjustments, e.g., the first adjustment 1404. Additional review 1424 may be implemented and may, in some cases, undo prior reviews and/or changes. In some implementations, an additional adjustment may be made before review of the prior adjustment 1404. Review may be implemented. In some implementations, an adjustment 1426 may not conflict with other adjustments but may still merit review 1428. For example, the system may automatically detect, by comparing the adjustment 1426 to a change log database, that the adjustment 1426 reverts a data element to a prior setting. The system may automatically flag such a change for review.

The collaboration system may support copying and comparing data from across multiple EHR systems via embedded translation layers that help map similar content together for human consideration in the approval process. In this, a “first change” may be the suggestion of adopting configuration settings from System A. A “second change” may be approval of adoption into System B by humans working for system B. Reporting and analytics to assist in identifying opportunities for standardizing best practices via this data and automatically action on it may be accomplished. For example, if most EHR systems that use the collaboration system have configured medication x to have dosing button y, there may be an opportunity to pull that configuration into the system with ease using an EHR configuration efficiency module.

The collaboration system may translate human-readable questions/answers to machine-readable configuration. For example, a question of, “Should a medication be delivered Stat?” can be set to automatically configure elements of an EHR to set a medication data element of an EHR to “stat,” mirroring an institutions workflow (across multiple data elements or singular). This layer of translation of human readable questions to build configuration effectively automates elements of work managed by informaticists in a health system, saving significant time and resources, and allowing for faster and more effective communication between institutions and/or different systems. This module may also encode standard procedures of configuration in change management workflows such that a decision can be mapped to an automated process. For example, some medications must have specific capitalization for safety benefits in identification (e.g., Tallman lettering). The system can be set to detect said instances of medication names and replace the content with appropriate casing.

A feature illustrated in one of the figures may be the same as or similar to a feature illustrated in another of the figures. Similarly, a feature described in connection with one of the figures may be the same as or similar to a feature described in connection with another of the figures. The same or similar features may be noted by the same or similar reference characters unless expressly described otherwise. Additionally, the description of a particular figure may refer to a feature not shown in the particular figure. The feature may be illustrated in and/or further described in connection with another figure.

Elements of processes (i.e. methods) described herein may be executed in one or more ways such as by a human, by a processing device, by mechanisms operating automatically or under human control, and so forth. Additionally, although various elements of a process may be depicted in the figures in a particular order, the elements of the process may be performed in one or more different orders without departing from the substance and spirit of the disclosure herein.

The foregoing description sets forth numerous specific details such as examples of specific systems, components, methods and so forth, in order to provide a good understanding of several implementations. It will be apparent to one skilled in the art, however, that at least some implementations may be practiced without these specific details. In other instances, well-known components or methods are not described in detail or are presented in simple block diagram format in order to avoid unnecessarily obscuring the present implementations. Thus, the specific details set forth above are merely exemplary. Particular implementations may vary from these exemplary details and still be contemplated to be within the scope of the present implementations.

Related elements in the examples and/or implementations described herein may be identical, similar, or dissimilar in different examples. For the sake of brevity and clarity, related elements may not be redundantly explained. Instead, the use of a same, similar, and/or related element names and/or reference characters may cue the reader that an element with a given name and/or associated reference character may be similar to another related element with the same, similar, and/or related element name and/or reference character in an example explained elsewhere herein. Elements specific to a given example may be described regarding that particular example. A person having ordinary skill in the art will understand that a given element need not be the same and/or similar to the specific portrayal of a related element in any given figure or example in order to share features of the related element.

It is to be understood that the foregoing description is intended to be illustrative and not restrictive. Many other implementations will be apparent to those of skill in the art upon reading and understanding the above description. The scope of the present implementations should, therefore, be determined with reference to the appended claims, along with the full scope of equivalents to which such claims are entitled.

The foregoing disclosure encompasses multiple distinct examples with independent utility. While these examples have been disclosed in a particular form, the specific examples disclosed and illustrated above are not to be considered in a limiting sense as numerous variations are possible. The subject matter disclosed herein includes novel and non-obvious combinations and sub-combinations of the various elements, features, functions and/or properties disclosed above both explicitly and inherently. Where the disclosure or subsequently filed claims recite “a” element, “a first” element, or any such equivalent term, the disclosure or claims is to be understood to incorporate one or more such elements, neither requiring nor excluding two or more of such elements.

As used herein “same” means sharing all features and “similar” means sharing a substantial number of features or sharing materially important features even if a substantial number of features are not shared. As used herein “may” should be interpreted in a permissive sense and should not be interpreted in an indefinite sense. Additionally, use of “is” regarding examples, elements, and/or features should be interpreted to be definite only regarding a specific example and should not be interpreted as definite regarding every example. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, Abstract, and any other document and/or resource incorporated herein by reference.

As used herein regarding a list, “and” forms a group inclusive of all the listed elements. For example, an example described as including A, B, C, and D is an example that includes A, includes B, includes C, and also includes D. As used herein regarding a list, “or” forms a list of elements, any of which may be included. For example, an example described as including A, B, C, or D is an example that includes any of the elements A, B, C, and D. Unless otherwise stated, an example including a list of alternatively-inclusive elements does not preclude other examples that include various combinations of some or all of the alternatively-inclusive elements. An example described using a list of alternatively-inclusive elements includes at least one element of the listed elements. However, an example described using a list of alternatively-inclusive elements does not preclude another example that includes all of the listed elements. And, an example described using a list of alternatively-inclusive elements does not preclude another example that includes a combination of some of the listed elements. As used herein regarding a list, “and/or” forms a list of elements inclusive alone or in any combination. For example, an example described as including A, B, C, and/or D is an example that may include: A alone; A and B; A, B and C; A, B, C, and D, and so forth. The bounds of an “and/or” list are defined by the complete set of combinations and permutations for the list.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not have been redundantly labeled. A person having ordinary skill in the art will recognize based on the disclosure herein redundant and/or duplicated elements of the same FIG. Despite this, redundant labeling may be included where helpful in clarifying the structure of the depicted examples.

The Applicant(s) reserves the right to submit claims directed to combinations and sub-combinations of the disclosed examples that are believed to be novel and non-obvious. Examples embodied in other combinations and sub-combinations of features, functions, elements and/or properties may be claimed through amendment of those claims or presentation of new claims in the present application or in a related application. Such amended or new claims, whether they are directed to the same example or a different example and whether they are different, broader, narrower or equal in scope to the original claims, are to be considered within the subject matter of the examples described herein.

1-6. (canceled)

7. A system for electronic health record (EHR) collaboration, comprising:
an EHR data correlation module comprising comparison logic and an EHR change log database, wherein:
EHR data is received by the EHR data correlation module from an EHR database, the EHR data indicative of an EHR;
the EHR data is compared, by the EHR data correlation module, to prior version data associated with the EHR data, wherein the prior version data is stored in the EHR change log database; and
in response to the EHR data differing from the prior version data:
the prior version data is updated by the EHR data correlation module to match the EHR data; or
new prior version data is generated by the EHR data correlation module to match the EHR data;
a roles module comprising a user database that correlates a user with a role;
a workflow module comprising logic associated with a workflow by which EHR data is updated, wherein:
update data corresponding to an update to the EHR data is received via the workflow module from a first user associated with a first role indicated by the roles module;
the update data is handled via the logic associated with the workflow; and
the workflow comprises review trigger logic; and
a review cycle module comprising logic associated with a review cycle by which the update to the EHR data via the workflow module is reviewed by one or more additional users, each of the one or more additional users having one or more different roles from the first user, wherein:
the review trigger logic of the workflow module triggers notification logic of the review cycle by which a second user is notified of the update to the EHR data; and
update logic of the review cycle module exports the update data to the EHR database or updates the EHR data according to the update data.

8-9. (canceled)

10. The system of claim 7, wherein the first user is associated with the first role, the first role comprising one or more permissions to propose updates to the EHR data via the workflow module, and wherein the second user is associated with a second role different from the first role.

11. The system of claim 7, wherein the update data corresponding to the update to the EHR data comprises proposed changes to patient medical information, medication data, treatment protocols, or laboratory test results stored in the EHR database.

12. The system of claim 7, wherein the update logic of the review cycle module exports the update data to the EHR database after the second user approves the update data during the review cycle.

13. The system of claim 7, wherein the notification logic of the review cycle comprises generating notification data associated with the update to the EHR data and transmitting the notification data to a second user device associated with the second user.

14. The system of claim 7, wherein the comparison logic of the EHR data correlation module compares the EHR data to the prior version data by identifying differences between the EHR data and corresponding prior version data stored in the EHR change log database.

15. A device for electronic health record (EHR) collaboration, the device having implemented thereon:
an EHR data correlation module comprising comparison logic, wherein:
EHR data is received by the EHR data correlation module from an EHR database, the EHR data indicative of an EHR;
the EHR data is compared, by the EHR data correlation module, to prior version data associated with the EHR data, wherein the prior version data is stored in an EHR change log database; and
in response to the EHR data differing from the prior version data:
the prior version data is updated by the EHR data correlation module to match the EHR data; or
new prior version data is generated by the EHR data correlation module to match the EHR data;
a roles module that correlates a user indicated by a user database with a role indicated by the user database;
a workflow module comprising logic associated with a workflow by which the EHR data is updated, wherein:
update data corresponding to an update to the EHR data is received via the workflow module from a first user associated with a first role indicated by the roles module;
the update data is handled via the logic associated with the workflow; and
the workflow comprises review trigger logic; and
a review cycle module comprising logic associated with a review cycle by which the update to the EHR data via the workflow module is reviewed by one or more additional users, each of the one or more additional users having one or more different roles from the first user, wherein:
the review trigger logic of the workflow module triggers notification logic of the review cycle by which a second user is notified of the update to the EHR data; and
update logic of the review cycle module exports the update data to the EHR database or updates the EHR data according to the update data.

16. The device of claim 15, wherein the second user is associated with a second role indicated by the roles module, and wherein the second role is different from the first role.

17. The device of claim 15, wherein the update data corresponding to the update to the EHR data comprises one or more proposed changes to patient medical history, diagnoses, medications, treatment plans, immunization dates, allergies, radiology images, or laboratory test results.

18. The device of claim 15, wherein the update logic of the review cycle module exports the update data to the EHR database after the one or more additional users approve the update data during the review cycle.

19. The device of claim 15, wherein the notification logic of the review cycle comprises generating notification data identifying the update to the EHR data and transmitting the notification data to a second user device associated with the second user.

20. The device of claim 15, wherein the comparison logic of the EHR data correlation module compares the EHR data to the prior version data by identifying differences between current EHR data values and corresponding prior version data values stored in the EHR change log database.

21. The device of claim 15, wherein:
the update logic of the review cycle module exports the update data to the EHR database;
the EHR data correlation module receives the EHR data from the EHR database after the update data has been exported to the EHR database;
the comparison logic compares the EHR data to the prior version data stored in the EHR change log database;
in response to the EHR data differing from the prior version data, the new prior version data is generated by the EHR data correlation module to match the EHR data; and
the new prior version data is stored in the EHR change log database.

22. A method of electronic health record (EHR) collaboration, comprising:
receiving, by an EHR data correlation module from an EHR database, EHR data indicative of an EHR;
comparing, by the EHR data correlation module, the EHR data to prior version data associated with the EHR data, wherein the EHR data correlation module comprises an EHR change log database in which the prior version data is stored;
in response to the EHR data differing from the prior version data:
updating, by the EHR data correlation module, the prior version data to match the EHR data, or
generating, by the EHR data correlation module, new prior version data that matches the EHR data;
correlating, by a roles module, a first user with a role using a user database of the roles module;
receiving, via a workflow module, update data corresponding to an update to the EHR data, wherein the workflow module comprises logic associated with a workflow by which the EHR data is updated, wherein the workflow module further comprises review trigger logic;
updating the EHR data by a workflow module based on the update data, wherein the update data is handled via the logic associated with the workflow;
triggering, by the review trigger logic, notification logic of a review cycle module, wherein the review cycle module comprises logic associated with a review cycle by which the update to the EHR data via the workflow module is reviewed by one or more additional users, each of the one or more additional users having one or more different roles from the first user, wherein the notification logic of the review cycle notifies a second user of the update to the EHR data; and
exporting the update data to the EHR database or updating the EHR data according to the update data.

23. The method of claim 22, wherein the second user is associated with a second role indicated by the roles module, and wherein the second role is different from the first role.

24. The method of claim 22, wherein the update data corresponding to the update to the EHR data comprises one or more proposed changes to medication data, treatment protocols, preparation methods, dispensing methods, dosing protocols, or storage information.

25. The method of claim 22, wherein exporting the update data to the EHR database or updating the EHR data according to the update data is performed after the one or more additional users approve the update data during the review cycle.

26. The method of claim 22, wherein the notification logic of the review cycle comprises generating notification data indicating that the first user has proposed the update to the EHR data and transmitting the notification data to a second user device associated with the second user.

27. The method of claim 22, wherein comparing the EHR data to the prior version data comprises identifying, by the comparison logic of the EHR data correlation module, differences between the EHR data received from the EHR database and the prior version data stored in the EHR change log database.

28. The method of claim 22, wherein:
exporting the update data to the EHR database comprises transmitting the update data from the review cycle module to the EHR database;
the EHR data correlation module receives updated EHR data from the EHR database after the update data has been exported to the EHR database;
comparing the updated EHR data to the prior version data comprises comparing, by the comparison logic, the updated EHR data to the prior version data stored in the EHR change log database;
in response to the updated EHR data differing from the prior version data, generating the new prior version data comprises generating, by the EHR data correlation module, the new prior version data that matches the updated EHR data; and
the new prior version data is stored in the EHR change log database.
